(19) United States
(12) Patent Application Publication
(10) Pub. No.: US 2006/0083788 A1
(43) Pub. Date: Apr. 20, 2006

(54) Water containing active hydrogen and process for producing the same

(76) Inventor: Kei Usui, Musashimurayama-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/513,516

(22) PCT Filed: May 9, 2003
(86) PCT No.: PCT/JP03/05824

§ 371 Date: Aug. 25, 2005

(30) Foreign Application Priority Data
May 10, 2002 (JP) 2002-136163
Jun. 21, 2002 (JP) 2002-181351

(51) Int. Cl. A61K 33/38; A61K 33/24; A61K 33/00

(52) U.S. Cl. 424/600; 424/618; 424/649

(57) ABSTRACT

Provided are novel water containing active hydrogen wherein the content of active hydrogen is high and a process wherein such active hydrogen-containing water is produced simply at high efficiency from an inexpensive material without needing complex apparatus or special treating agents. The water containing active hydrogen of the present invention with respect to which, in an electron spin resonance spectrum pattern obtained by measuring under such a condition that immediately after generation treatment of hydrogen radicals, 25% by mass of 5,5-dimethyl-1-pyrroline-N-oxide is added to thereby stabilize hydrogen radicals, the intensities of peaks ascribed to hydrogen radicals occurring in the vicinity of 331.8 mT magnetic field strength and in the vicinity of 335.5 mT magnetic field strength are 0.03 or greater and 0.04 or greater, respectively, taking the intensity of peak ascribed to manganese as the standard sample is produced by bringing the starting water into contact with activated carbon carrying a water-insoluble ferric oxide hydrate after a magnetization treatment.

### TECHNICAL FIELD

The present invention relates to novel water containing active hydrogen having the effect of scavenging active oxygen which is known to have a serious influence on the physiological phenomenon of organisms and to a method for producing the same.

### BACKGROUND ART

Water containing active hydrogen is known to have the effect of scavenging active oxygen and to suppress a physiologically adverse influence on organisms due to active oxygen. Many methods for producing active hydrogen-containing water have been proposed so far, these methods including, for example, a method in which untreated common water is subjected to electrical or physical treatment such as electrolytic treatment and ultrasonic treatment and the like and a method in which untreated water is chemically treated by using an oxidizing agent or a reducing agent. However, in fact, many of these methods are not approved under Food Sanitation Law.

For example, among so-called electrolytic water (obtained by electrolysis of water admixed with common salt and the like), the use of water obtained on the cathode side by electrolysis in a diaphragm process (alkaline water which is said to contain active hydrogen) is not approved under Food Sanitation Law and therefore the direct use of this water for foods is not authorized.

Thus, in order to produce active hydrogen-containing water and to use it for foods without any legal problem, there is nothing for it but to add hydrogen specified as a natural additive to water as active hydrogen by using a physical method or to use, as processing aids, materials approved under Food Sanitation Law.

The inventor of the present invention has proposed methods in which natural water is brought into contact with a palladium alloy occluded with hydrogen to generate active hydrogen-containing water, which is then used for the growth of organisms (JP 09-010756 A) and for the improvement of quality of a foodstuff (WO 01/03522 A1).

These methods, however, necessitate specific devices or use of expensive treating agents to cause inevitably problems such as troublesome operations and high cost.

### DISCLOSURE OF THE INVENTION

The present invention has been made under such a situation for the purpose of providing novel active hydrogen-containing water containing active hydrogen in a high content and a method for the preparation thereof simply at high efficiency by using inexpensive materials without any complicated devices and special treating agents.

The inventor of the present invention has conducted various studies as to the production of active hydrogen-containing water repeatedly and, as a result, found that high-concentration active hydrogen-containing water having the effect of scavenging active oxygen is obtained in a simple operation with high efficiency by using activated carbon after a special treatment as a catalyst, to accomplish the present invention based on this finding.

Thus, the present invention provides water containing active hydrogen characterized in that, in the electron spin resonance (ESR) spectrum pattern as determined in a condition that a hydrogen radical-generating treatment is immediately followed by the addition of 25% by mass of 5,5-dimethyl-1-pyrroline-N-oxide to effect stabilization of the hydrogen radicals, the ESR peaks originating in the hydrogen radicals appearing in the vicinity of 331.8 mT and in the vicinity of 335.5 mT of the magnetic field strength have intensities of at least 0.03 and at least 0.04, respectively, taking the intensity of the peak originating in manganese used as the standard sample as 1, and a method for the preparation of the active hydrogen-containing water wherein the starting water is brought into contact with an activated carbon catalyst carrying a water-insoluble iron(III) oxide hydrate after a magnetization treatment with or without a noble metal catalyst, as the case may be.

### BRIEF DESCRIPTION OF THE DRAWINGS

FIG. 1 is the ESR spectrum pattern of the active hydrogen-containing water according to the invention.

FIG. 2 is the ESR spectrum pattern of untreated tap water.

FIG. 3 is the ESR spectrum pattern of conventional active water.

### BEST MODE FOR CARRYING OUT THE INVENTION

The present invention will be hereinafter explained in detail.

The active hydrogen-containing water of the present invention contains active hydrogen in an outstandingly higher concentration than active water produced by a conventional method and this fact can be confirmed easily by measuring the electron spin resonance spectrum of the water.

Various methods are known so far as a method for the preparation of water containing active hydrogen as mentioned above. However, hydrogen radicals are very unstable and disappear in a short time so that the existence thereof is confirmed merely qualitatively but not quantitatively.

In order to measure the concentration of active hydrogen quantitatively, the inventor of the present invention treated the starting water to generate hydrogen radicals, then, as soon as possible, added a trapping agent, for example, 5,5-dimethyl-1-pyrroline-N-oxide (hereinafter referred to as DMPO) and froze the mixture quickly by using a cooling medium, for example, liquid nitrogen, to trap hydrogen radicals, thereby to measure the ESR spectrum. Thus, the inventor succeeded in measuring hydrogen radicals quantitatively on the basis of the relative intensities of the hydrogen radicals in the obtained spectrum pattern.

The active hydrogen-containing water of the present invention is clearly different from conventional active water in the point that the hydrogen radicals measured quantitatively in the above manner have high concentration such that the peaks originating in the hydrogen radicals appearing in the vicinity of 331.8 mT and in the vicinity of 335.5 mT of the magnetic field strength have intensities of at least 0.03 or, particularly, at least 0.1 and at least 0.04 or, particularly, at least 0.2, respectively, of the intensity of the peak originating in manganese used as the standard sample.

In the case of active water obtained using a hitherto known palladium catalyst, on the other hand, the peaks originating in the hydrogen radicals and appear in the vicinity of 331.8 mT and in the vicinity of 335.5 mT of magnetic field strength, when measured in the same method, have intensities of 0.023 and 0.035 of the intensity of the peak originating in manganese, respectively. In the case of active water produced by using a commercially available product of so-called active water-producing apparatuses based on various principles, absorption of hydrogen radicals is hardly found.

The reason why the peak at a position of 331.8 mT of magnetic field strength is selected is that there is no fear that this peak overlaps on the peaks of other radicals while the reason why the peak at a position of 335.5 mT is selected is that the peak of the hydrogen radicals reaches a maximum within a range from 330 to 340 mT of the scanning range of the magnetic field to be used.

Generally, hydrogen radicals have lower reactivity than hydroxylradicals and the like. Therefore, in order to completely trap these hydrogen radicals, it is preferable to add a trapping agent, for example, DMPO, in an amount as large as possible, i.e. in an amount up to about 25% by mass.

While the absolute value of the intensity in an ESR spectrum corresponding to each component varies according to factors such as the type of detector, the conditions of measurement such as microwave output, magnetic field scanning width, scanning time, magnetic field modulation and magnetic field strength and the amount of a trapping agent, the relative intensity of the peak originating in these hydrogen radicals appearing in the vicinity of 331.8 mT or in the vicinity of 335.5 mT of the specific magnetic field strength to the peak originating in manganese used as the standard sample is independent of the above factors and always indicates reproducible values.

The active hydrogen-containing water containing hydrogen radicals in such a high concentration according to the present invention is prepared by bringing the starting water into contact with an activated carbon carrying a water-insoluble ferric oxide hydrate after a magnetization treatment or an activated carbon carrying a water-insoluble ferric oxide hydrate after a magnetization treatment and a noble metal catalyst.

As the activated carbon to be used in this case, those having a low content of impurities are used among those hitherto used as an activated carbon for adsorption purpose. However, it is the rule to use an activated carbon which is highly safe using, as the starting material, particularly, vegetable origin wood flours, sawdusts, coconut shells and pulp powders, namely, activated carbon satisfying the requirements as to safety prescribed in the City Water Law and the Food Sanitation Law.

However, it is possible to use, as required, activated carbon obtained by using mineral type starting materials such as coal, petroleum residue, petroleum cokes and petroleum pitch or using plastic resins such as phenol resins, furan resins, urea resins, polyvinyl chloride, polyvinylidene chloride and polycarbonate. Such activated carbon may be used after activation by zinc chloride or phosphoric acid according to need.

As this activated carbon, preferable are those having a pore diameter of 2 to 100 nm and a specific surface area measured by BET method of at least 200 m²/g or, preferably, 500 to 1500 m²/g. This activated carbon is used in the form of granules having an average particle diameter of 0.2 to 1.5 mm.

In the method of the present invention, it is required that a water-insoluble ferric oxide hydrate is subjected to a magnetization treatment and, simultaneously, carried on the activated carbon. The water-insoluble ferric oxide hydrate in this case is a compound having a composition represented by the general formula Fe₂O₃.xH₂O or FeO(OH).

This water-insoluble ferric oxide hydrate per se is produced through process including hydrolysis of Fe(III) ions, polymerization and production of a water-insoluble hydrate in a pH falling in a neutral region. As this Fe(III) ion source, those approved under the Food Sanitation Law such as, for example, ferric chloride and the like are preferable.

This activated carbon catalyst is obtained by adsorbing iron ions to the starting activated carbon followed by hydration polymerization by using iron ions as nuclei and fixation through each of the above steps. When an external magnetic field is applied in this process, Fe³⁺ causes electron spin resonance (ESR) because it is a paramagnetic ion. The hydrate polymer having Fe as the nucleus changes in state and, as a result, an activated carbon catalyst having strong activity is obtained.

Fe³⁺ ions are caused to act on the pore portions on the surface of the activated carbon by utilizing the above phenomenon to effect binding of free radicals on the surface with Fe³⁺. In the succeeding process, an external magnetic field is applied, hydration polymerization is run using Fe³⁺ fixed to the surface of activated carbon as a nucleus with maintaining the ESR state by applying an electromagnetic field having a resonance frequency to make the hydrate polymer insoluble in water while maintaining the system in a free-radical-rich state differing from the ordinary state.

In other worlds, the object of the ESR which is normally utilized to detect a superfine or fine structure is diverted reversely to the object of changing the position or state of an unpaired electron in a molecule to control its radical structure.

Thus, by using a device such as that used in an ESR measuring instrument which device has both the ability to change a magnetic field strength by an electromagnet and the ability to radiate microwave, a magnetic field, for example, in the vicinity of 330 mT (millitesla) is applied and a Fe³⁺ solution which has been prepared in advance is brought into contact with an activated carbon with applying microwave having an appropriate resonance frequency of up to 35 GHz as the maximum to expedite binding of the surface of the activated carbon with Fe and the subsequent hydration polymerization.

It is necessary to adjust each condition in this case according to the characteristics such as the amount of free radicals required for an activated carbon catalyst, i.e., reactivity. Even if Fe is bound with the surface of the activated carbon and the subsequent hydration is not completed, deprotonation dissociating H⁺ (proton) from an aquocomplex proceeds. Even if the external magnetic field is removed at the juncture wherein the pH rises to the neutrality, its effect is kept and therefore it is only required to apply the external magnetic field in the initial stage.

Therefore, when the pH rises to the neutral region, the application of the external magnetic field and the radiation of microwave are terminated and the system is kept standing for further at least 24 hours to accomplish aging. At this time, the system is dried under heating to a temperature of 40° C. or more and less than 100° C. under normal pressure to promote a dehydration reaction and the fixing, treatment is terminated.

24 hours or more must be taken usually for this drying and fixing treatment though the time differs depending on various conditions such as temperature.

Even after completion of the drying, the mass amount of the system increases because a hydration polymer is produced in an amount equivalent to 10% or more of the initial mass of the activated carbon.

Moreover, even in the case of measuring a magnetic field by a simple method, common activated carbon has a magnetic field of only 0.01 mT or less in a DC magnetic field. However, the activated carbon catalyst to which a hydration polymer is added has a magnetic field of 0.02 to 0.05 mT or more.

The active hydrogen-containing water of the present invention has the effect of scavenging active oxygen. This can be confirmed by utilizing the fact that this is accompanied by a faint emission phenomenon when active oxygen reacts with a reducing material and by measuring the intensity of the emitted light. This method can be performed according to the method disclosed in “Luminescence 2001” published by John Willy & Sons, Vol. 16, pp 1-9, 2001, Report “Imaging of hydroperoxide and hydrogenperoxide-scavenging substances by photon emission”, wherein an emission test of XYZ system active oxygen scavenging is conducted to measure the luminescence of the Y-component. In this method, X means active oxygen, Y means a scavenger (hydrogen donor) and Z means a catalyst.

In the method of the present invention, as is mentioned above, the water-insoluble ferric oxide hydrate after a magnetization treatment is carried by the activated carbon thereby to improve the electron-donating power of the activated carbon. As a result, dissociation of water is promoted and hydrogen constituting a part of the water molecule is reduced and released as active hydrogen into water to produce active hydrogen-containing water. When active oxygen is present, the active hydrogen scavenges the active oxygen by reacting therewith.

Generally, activated carbon originally has the ability of dehydrogenating hydrocarbons and the like, which ability is, however, by no means high. Usually, the dehydrogenation proceeds only when oxygen or other hydrogen acceptors exist. However, when the catalyst is made to carry various transition metals, not only dehydrogenation activity is outstandingly improved but also the hydrogen adsorbing ability of the activated carbon is increased from several tens times to several hundreds times that of the adsorbed metals due to synergetic effect. Then, adsorbed hydrogen molecules are dissociated on the surface of the metals to be an atomic state and retained on the activated carbon. The hydrogen on this activated carbon is dissociated quickly in, for example, medium water to form active hydrogen-containing water.

On the other hand, it is known that, when a noble metal is carried on an activated carbon, the catalytic activity of the activated carbon is significantly improved. It is therefore preferable that a noble metal catalyst is carried on the activated carbon for the treatment of the present invention. As the noble metal catalyst, for example, platinum, palladium or silver is used. The amount of the noble metal to be carried is in a range from 0.07 to 3 ppm or, preferably, 0.1 to 1 ppm based on the mass of the activated carbon.

The preparation of the active hydrogen-containing water according to the present invention is conducted in the following manner that the activated carbon catalyst carrying the water-insoluble ferric oxide hydrate after a magnetization treatment or a mixture of the water-insoluble ferric oxide hydrate after a magnetization treatment and a noble metal catalyst is taken to fill a column to pass the starting water at a rate of SV of at least 10 or, preferably, 20 to 30. At this time, it is advantageous to adopt a system using a cartridge which can be equipped with the column in a dismountable manner and is filled with the activated carbon catalyst instead of filling the column directly with the activated carbon catalyst, because the activated carbon catalyst can be easily exchanged when its catalytic activity has been reduced.

The present invention is explained in more detail by way of examples, which, however, do not limit the present invention.

### REFERENCE EXAMPLE (PREPARATION OF ACTIVATED CARBON CATALYST)

A 100 g portion of activated carbon (average particle diameter of 1.00 mm and specific surface area of 1350 m²/g) was dipped in 500 ml of an aqueous ferric chloride solution having a concentration of 1 mol and 700 ml of an aqueous ammonium carbonate solution having a concentration of 1 mol were added dropwise to the solution. Then, the solution was placed in a DC magnetic field of 323 mT and heated at 60° C. for 30 minutes under radiation of microwave having a resonance frequency. Then, the activated carbon was separated by filtration and heated at 100° C. for 10 hours to obtain 121 g of an activated carbon catalyst carrying a water-insoluble ferric oxide hydrate after a magnetization treatment (hereinafter referred to as the magnetic activated carbon).

### Example 1

A 300 g portion of the magnetic activated carbon obtained in the same manner as in Reference Example was taken to fill a glass column (inner diameter of 60 mm and length of 200 mm), through which tap water was passed at a SV of 20 to conduct activating treatment, thereby to produce active hydrogen-containing water.

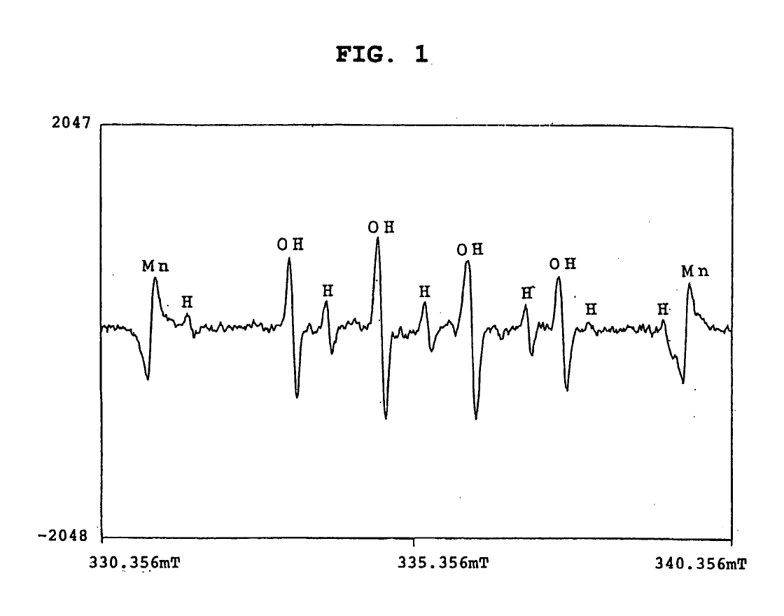

10 ml of the active hydrogen-containing water were taken in a sample tube, to which DMPO was first added in such a way that the concentration thereof reached 1% by mass followed by mixing and the sample tube was dipped immediately in liquid nitrogen to freeze the mixture. Then, the mixture was thawed at room temperature to measure the ESR spectrum by an ESR measuring instrument (product name: “Type JES-FA200”, manufactured by Nippon Denshi Co.) in the following conditions: microwave output: 8 mW, magnetic field scanning range: 335 mT±5 mT, scanning time: 2 minutes and magnetic field modulation: 100 kHz, to find no peaks. Separately, DMPO was added to the mixture until the concentration had reached 25% by mass to measure the ESR spectrum in the same manner. The spectrum pattern obtained in the above manner is shown in FIG. 1. In the graph showing the pattern, the abscissa is for the strength (mT) of the magnetic field and the ordinate is for the relative intensity.

As is understood from this figure, peaks originated in the hydrogen radicals were found at the positions of the magnetic field strength of 331.8 mT, 334.0 mT, 335.5 mT, 337.2 mT, 338.1 mT and 339.3 mT and the peak at the position of 335.5 mT exhibited a maximum value.

The relative values of these peaks to that of the standard sample Mn are shown in Table 1.

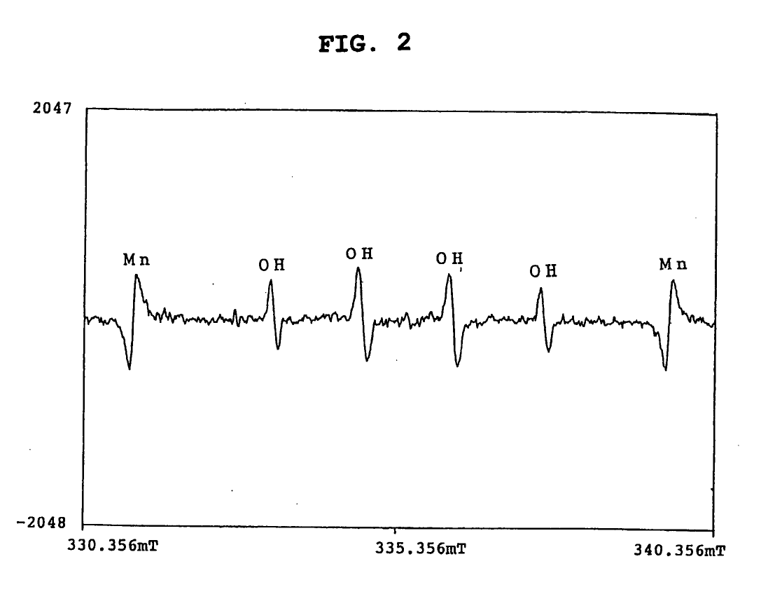

For comparison, the ESR spectrum pattern of untreated tap water is shown in FIG. 2 and the relative values of the peaks originated in the hydrogen radicals to that of the standard sample Mn are also shown in Table 1.

### Comparative Example 1

A reactor having a honeycomb structure was prepared by filling a stainless steel-made reactor tube having an inner diameter of 150 mm and a length of 300 mm with 114 chips of small hard-plastic cylinders having an outer diameter of 25 mm, wall thickness of 3 mm and length of 50 mm and provided with a 2 μm thick Pd metal film on the inner and outer surfaces.

This reactor was kept in a dry state and the air in the reactor was completely replaced with hydrogen gas. Then, the reactor was kept under a hydrogen pressure of 0.8 MPa at 15° C. for 10 minutes to allow the above Pd metal film to occlude hydrogen. Next, the supply of hydrogen gas was stopped and 5 liters of distilled water were immediately introduced to the reactor and then discharged after the reactor was kept standing for 5 minutes, to obtain active water.

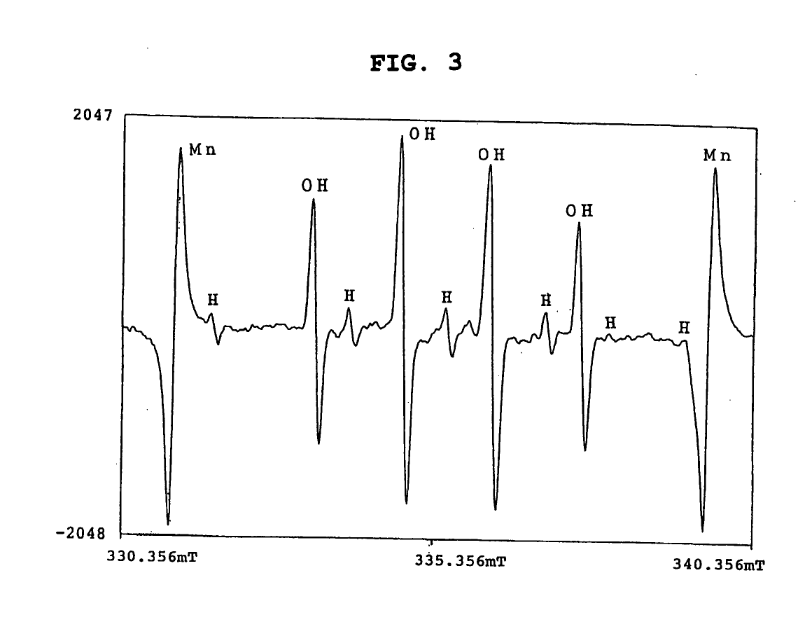

The ESR spectrum of the active water obtained in this manner was measured in the same method as in Example 1. The results are shown in FIG. 3. The relative value of each ESR peak of hydrogen radicals in this figure to the standard Mn is shown in Table 1.

TABLE 1

| | Standard sample | Hydrogen radicals (mT) | | | | | | | |
|---|---|---|---|---|---|---|---|---|---|
| | Mn | 331.8 | | 335.5 | | | | | |
| Sample | (X) | (Y) | 334.0 | (Z) | 337.2 | 338.1 | 339.3 | Y/X | Z/X |
| Example 1 | 23.0 | 5.5 | 9.8 | 9.8 | 8.9 | 2.6 | 3.8 | 0.24 | 0.43 |
| Comparative | 75.1 | 1.7 | 2.4 | 2.6 | 2.3 | 0.2 | 0 | 0.023 | 0.035 |
| Example 1 | | | | | | | | | |
| Untreated | 18.0 | 0 | 0 | 0 | 0 | 0 | 0 | 0 | 0 |
| tap water | | | | | | | | | |

As is clear from this table, the active hydrogen-containing water of the present invention contains hydrogen radicals in an outstandingly higher concentration than conventional active water.

### Comparative Example 2

The ESR spectrum of alkaline water obtained by using a commercially available alkali ion water conditioner was measured in the same manner as in Example 1. However, in the obtained ESR spectrum pattern, the peak of hydrogen radicals was not found at all.

### Example 2

A 300 g portion of the activated carbon catalyst obtained in the same manner as in Reference Example was taken to fill a column cylinder (inner diameter of 60 mm and length of 200 mm), through which tap water was passed at a SV of 20 to conduct an activating treatment, thereby to produce active hydrogen-containing water.

Next, an aqueous FeCl₂ solution was added to aqueous 3% by mass hydrogen peroxide to generate hydroxylradicals. Then, using the above active hydrogen-containing water, the power of scavenging hydroxylradicals was measured by the ESR spectrum method.

For comparison, the oxidation resistance of each of distilled water and commercially available ultra-pure water used for ESR was also measured. Using tap water as a control and taking its hydroxylradical scavenging power as 0, the relative value of the hydroxylradical scavenging power of each water sample was calculated.

As a result, the hydroxylradical scavenging power of each of distilled water and ultra-pure water was 6.25% and 20.5%, respectively, whereas the hydroxylradical scavenging power of the active hydrogen-containing water was 23.2%.

### Example 3

By utilizing the XYZ-system active oxygen-scavenging and luminescence method, measurement was made for the luminescence intensity of the Y-component indicating the oxidation resistance for a green tea (a tea bag product) extract with the active hydrogen-containing water obtained in Example 2.

As the measuring apparatus, “AQUACOSMOSNIM Micro-system” (manufactured by Hamamatsu Photonics Co.) was used. An aqueous 2% by mass hydrogen peroxide was used as the X reagent and an aqueous 10% by mass acetaldehyde solution saturated with potassium hydrogencarbonate was used as the Z reagent.

As the sample, a solution obtained in the following manner was used: 50 ml of the active hydrogen-containing water (pH 7.2) kept at a temperature of 70° C. or 15° C. was taken in a beaker and the tea bag was dipped in the water, kept standing for 90 seconds and moved up and down 5 times repeatedly to obtain an extract solution. The results of the test are shown in Table 2.

For comparison, the result of measurement for the tap water (pH 7.2) at 70° C. is also shown in Table 2.

TABLE 2

| | Luminescence | |
|---|---|---|
| | intensity of | Intensity ratio to |
| Water used | Y-component | tap water |
| Active hydrogen- | 410 | 1.52 |
| containing water (70° C.) | | |
| Active hydrogen- | 500 | 1.85 |
| containing water (15° C.) | | |
| Tap water (70° C.) | 270 | 1 |

### Example 4

A 5 g portion of a commercially available coffee powder was placed in a coffee dripper and 50 ml of the active hydrogen-containing water obtained in Example 2 and kept at 70° C. was poured onto the coffee powder. The sample was kept standing for about one minute and subjected to the same test as in Example 2 to measure the luminescence intensity of the Y-component. The result is shown in Table 3. For comparison, the result of measurement of the tap water (pH 7.2) at 70° C. is also shown in Table 3.

TABLE 3

| | Luminescence | |
|---|---|---|
| | intensity of | Intensity ratio to |
| Water used | Y-component | tap water |
| Active hydrogen- | 1900 | 1.73 |
| containing water | | |
| Tap water | 1100 | 1 |

It is understood from the above results that the active hydrogen-containing water of the present invention has significantly higher oxidation resistance than the tap water.

### Example 5

A lettuce-browning prevention test was conducted by using active hydrogen-containing water. The lettuce browning reaction is considered to be a phenomenon that polyphenols such as non-colored catechol contained in lettuce are oxidized by oxygen contained in the atmosphere and converted into brown-colored substances.

The activated carbon catalyst obtained in Reference Example was taken to fill a glass column (inner diameter of 100 mm and length of 300 mm), through which well water (pH 7.5) was passed at a SV of 20 to obtain active hydrogen-containing water, which was then used as the sample.

The active hydrogen-containing water (18° C.) obtained in this manner was supplied at a water supply rate of 10 l/minute to a cut vegetable washer (four 200 litters-capacity washing tanks in-series type) such that washing time in each tank was 2 minutes to wash lettuce. Then, the lettuce was dewatered for one minute by centrifugation at a rotation of 500 rpm and then packaged with an oxygen non-permeable nylon sheet with or without nitrogen sealing. After that, the packaged lettuce was stored under cooling at 8° C.

The occurrence of browning in the lettuce stored in this manner for 1 to 6 days was visually observed. The results are shown in Table 4. For comparison, the results obtained by using untreated well water are also shown.

TABLE 4

| | | |
|---|---|---|
| Days of | | Active hydrogen- |
| storage | Well water | containing water |

| (days) | with N₂ | without N₂ | with N₂ | without N₂ |
|---|---|---|---|---|
| 1 | A | A | A | A |
| 2 | A | A | A | A |
| 3 | B | B | A | A |
| 4 | B | B | B | A |
| 5 | C | C | B | A |
| 6 | C | C | B | A |

The symbols for evaluation in the above table have the following meanings.
A: Not browned
B: Partly browned
C: All browned

As is understood from this table, browning was found on the lettuce washed with well water already on the third day whereas no browning was found on the lettuce washed with the active hydrogen-containing water and stored without nitrogen sealing even after 6 days.

### INDUSTRIAL APPLICABILITY

According to the present invention, active hydrogen-containing water in high concentration is provided by using a simple apparatus and the obtained active hydrogen-containing water can be widely used for storage of fresh foods, sterilization, drinking water and growth of organisms like conventional active water and exhibits more excellent effects. Also, the use of the water makes it possible to efficiently prevent environmental disruption and inhibition to the health of various organisms caused by active oxygen.

1. Active hydrogen-containing water characterized in that, in the electron spin resonance spectrum pattern as determined in a condition that a hydrogen radical-generating treatment is immediately followed by the addition of 25% by mass of 5,5-dimethyl-1-pyrroline-N-oxide to effect stabilization of the hydrogen radicals, the peaks originating in the hydrogen radicals appearing in the vicinity of 331.8 mT and in the vicinity of 335.5 mT of the magnetic field strength have intensities of at least 0.03 and at least 0.04, respectively, taking the intensity of the peak originating in manganese used as the standard sample as 1.

2. A method for the preparation of the active hydrogen-containing water described in claim 1 characterized in that the starting water is brought into contact with an activated carbon carrying a water-insoluble ferric oxide hydrate after a magnetization treatment.

3. A method for the preparation of the active hydrogen-containing water described in claim 1 characterized in that the starting water is brought into contact with an activated carbon carrying a noble metal catalyst and a water-insoluble iron(III) oxide hydrate after a magnetization treatment.

4. The method for the preparation of active hydrogen-containing water described in claim 2 in which the activated carbon has a specific surface area of at least 200 m²/g.

5. The method for the preparation of active hydrogen-containing water described in claim 3 in which the metallic element of the noble metal catalyst is platinum, palladium or silver.

6. The method for the preparation of active hydrogen-containing water described in claim 3 in which the activated carbon has a specific surface area of at least 200 m²/g.
